(19) United States
(12) Patent Application Publication
(10) Pub. No.: US 2013/0171278 A1
(43) Pub. Date: Jul. 4, 2013

(54) COMPOSITION FOR PROMOTING MEMORY AND LEARNING ABILITY

(71) Applicants: Chang-Ho Lee, Yongin-si (KR); In-Ho Kim, Yongin-si (KR); Dae Seok Han, Seongnam-si (KR); Young-Eon Kim, Yongin-si (KR); Dong-Jun Park, Yongin-si (KR); Jun-Hyeok Choi, Daegu (KR); Hye-Sung Lee, Seoul (KR); Sueong-Mock Cho, Seongnam-si (KR)

(72) Inventors: Chang-Ho Lee, Yongin-si (KR); In-Ho Kim, Yongin-si (KR); Dae Seok Han, Seongnam-si (KR); Young-Eon Kim, Yongin-si (KR); Dong-Jun Park, Yongin-si (KR); Jun-Hyeok Choi, Daegu (KR); Hye-Sung Lee, Seoul (KR); Sueong-Mock Cho, Seongnam-si (KR)

(73) Assignee: KOREA FOOD RESEARCH INSTITUTE, Seongnam-si (KR)

(21) Appl. No.: 13/822,714

(22) PCT Filed: Sep. 16, 2011
(86) PCT No.: PCT/KR11/06853

§ 371 Date: Mar. 13, 2013

(30) Foreign Application Priority Data
Sep. 17, 2010 (KR) 10-2010-0092028
Sep. 17, 2010 (KR) 10-2010-0092038
Sep. 17, 2010 (KR) 10-2010-0092045
Aug. 8, 2011 (KR) 10-2011-0078568

(51) Int. Cl. A61K 36/23; A61K 36/31; A61K 36/79

(52) U.S. Cl. CPC A61K 36/23; A61K 36/79; A61K 36/31
USPC 424/740; 424/769; 424/725

(57) ABSTRACT

The present invention relates to a composition for promoting memory or learning ability and to a composition for preventing and treating cognitive impairment, which comprise one, two or more plant extracts selected from the group consisting of Artemisia apiaceae extract, Illicium verum extract and Lepidium apefalum extract. The present invention has the effect of inhibiting neural cell damage, especially, neural cells of the basal part of the cerebrum by inhibiting acetylcholinesterase activity and through antioxidative activity (for example, inhibiting the generation of oxidative reactive species) and affinity for an NMDA receptor. The compositions of the present invention may have the effect of not only promoting memory or learning ability by protecting neural cells and preventing damage to neural cells but also of preventing and treating diseases caused by cognitive impairment. In addition, according to the present invention, Lepidium apefalum extract is provided as basic substance for pharmaceuticals or food which has efficacy in promoting memory or learning ability or efficacy for preventing and treating cognitive impairment.

### FIELD OF THE INVENTION

The present invention relates to compositions for enhancing memory and learning ability including an extract of Artemisia apiaceae, an extract of Illicium verum or an extract of Lepidium apefalum as active ingredients.

### DESCRIPTION OF THE RELATED ART

Each of 100 billion neurons present on human brain is connected to other neurons around it with synapses of average 10,000 to form a network each other.

Neurons closely communicate with each other through these synapses. At this time, neurotransmitters play the role of intermediation. Neurons release neurotransmitter to stimulate receptors such that they exchange information with each other.

The way in which human brain store memories is the process of reinforcement for synaptic connection strength through persistent chemical and electrical stimulations and it is called LPT (long-term potentiation). This property of synapse that neurotransmission networks between synapses are strengthened through persistent stimulations is synaptic plasticity.

The fact has been experimentally demonstrated in the early 20th century that information transmission in synapse is accomplished by neurotransmitters. Neurotransmitters currently known are acetylcholine (ACh), norepinephrine, dopamine (DA), GABA, glycine and glutamic acid (Table 1).

TABLE 1

| Type | Neurotransmitter | Presented region | Function | Related diseases | Related drugs |
|---|---|---|---|---|---|
| Amine | Acetylcholine | Autonomic nervous | Gland | Peptic | Antiulcer drug |
| | (ACh) | system, | secretion (liver, | ulcer, | (pirenzepine). |
| | | Midbrain nervous | secretion of | Dementia | Antidementia |
| | | system | digestive | | drug |
| | | | juice), | | |
| | | | Contraction | | |
| | | | and relaxation | | |
| | | | of smooth | | |
| | | | muscle and | | |
| | | | skeletal | | |
| | | | muscle, | | |
| | | | memory | | |
| Amine | serotonin | Midbrain nervous | Emotion, Sleep | Emotional | Antidepressant |
| | (5-HT) | system | | disturbance, | (imipramine) |
| | | | | Hallucination | |
| Catecholamine | Dopamine | Midbrain nervous | Consciousness, | Schizophrenia, | Antipsychotic |
| | (DA) | system (corpus | Movement | Parkinson's | agent |
| | | striatum, nucleus | | disease | (haloperidol), |
| | | accumbens) | | | Antiparkinsonism |
| | | | | | drug (L- |
| | | | | | dopa) |
| Catecholamine | Norepinephrine | Autonomic nervous | Contraction of | Emotional | Antidepressant |
| | (NA) | system | smooth muscle | disturbance | (desipramine) |
| | | (sympathetic | and | | |
| | | nerve), Midbrain | myocardium, | | |
| | | nervous system | Emotion, | | |
| | | | Consciousness, | | |
| | | | Appetite | | |
| Amino | Glycine | Midbrain nervous | Functional | Convulsion | Convulsion |
| acid | (Gly) | system | control of | | drug |
| | | | motor nerve | | (strychine) |
| Amino | GABA | Midbrain nervous | Sleep, | Convulsion | Sleeping drug |
| acid | | system | Skeletal | | (barbital), |
| | | | muscle tension | | Tranquilizer |
| | | | | | (benzodiazepine), |
| | | | | | Muscle |
| | | | | | relaxant |
| | | | | | (baclofen) |
| Amino | Glutamic | Midbrain nervous | Memory | Neural | Anticonvulsion |
| acid | acid | system | | death | drug, |
| | | | | | Antineural |
| | | | | | death drug |
| Polypeptide | Substance P | Midbrain nervous | Pain sense | | Antiinflammatory |
| | | system | transmission, | | drug |
| | | | Contraction of | | |
| | | | intestinal tract | | |
| Polypeptide | Enkephalin | Midbrain nervous | Pain sense | | Painkiller |
| | (Enk) | system | suppression, | | (morphine) |
| | | | Contraction | | |
| | | | suppression of | | |
| | | | intestinal tract | | |
| Gas | Nitrogen | Intestine, | Vasodilatation, | Neural | Antianginal |
| | monoxide | Midbrain nervous | Memory | death | drug |
| | (NO) | system (cerebellum | | | (nitroglycerin) |
| | | etc.) | | | |

The fact has been experimentally demonstrated in the early 20th century that information transmission in synapse is accomplished by neurotransmitters. Neurotransmitters currently known are acetylcholine (ACh), norepinephrine, dopamine (DA), GABA, glycine and glutamic acid (Table 1).

Studies have found that cognitive disorders such as loss of memory and learning ability caused by Alzheimer's disease (a kind of senile dementia) result from damage of acethylcholinergic neurons in cerebral base. Based on the studies, drugs capable of strengthening neuronal function via various mechanisms, for example, agonists for muscarine acetylcholine receptor, acetylcholine generation accelerators and acetylcholinesterase inhibitors have been developed. In the present, tacrine which is developed as acetylcholinesterase inhibitor has the gastrointestinal-related side effects. As the drugs show limitations in side effects, inhibitory activity substances derived from natural plant sources are drawing attentions.

Artemisia apiaceae Herba is a biennial herb which commonly grows south of central Korea. It is 40-150 cm tall with many twig in stem. Origin is whole part of Artemisia apiacea HANCE. It is taken before the summer flowering and dried in the shade to use. Main ingredients are abrotamine, vitamin A, β-bourbonene, caryophyllene, α-pinene, β-pinene, 1,8-cineole, α-thujone. It was reported that the plant has various pharmacological effects, including haemostasis, alleviating fever and inhibiting malarial parasites.

Illicium verum Hook f. is a fruit belongs to magnoliaceae and grows in tropical Asia. Its name, star anise, was named from the star-shaped fruit. The plant has promoting digestion effect. It is 1-2 cm tall with diameter 0.3-0.5 cm. The exterior is reddish brown and has irregular wrinkles. The inside is pale brown, soft and shiny. The length of the stem of the fruit is 3-4 cm and the stem is connected to the bottom. The length of seed of the fruit is 6 mm and the seed is reddish-brown or yellowish brown. The plant has a fragrance, sweet taste. The fruit includes about 5% of oil refining and 80-90% of the oil refining is anethole. In addition, it contains pinene, phellandren, cineol, limonene, dipentene, sophorol, anisketone, anisaldehyde and anisic acid.

Lepidium apefalum wild. is spread throughout Korea, central asia, himalayas, China. The plant is native to North America and grows in field. Its seed is brown, small disk-shaped with white membranous wings at the edge. The dried plant includes fatty oils, Sinigrin and sugars. The plant is used for cough, asthma, tuberculosis, exudative pleuritis, swelling, urinary retention and cardiac weakness in oriental medicine.

Flavonoids include isoflavones which are found in soybean, flavanols which are found in tea, cocoa and red wine, and anthocyanins. The mechanism of isoflavones is to mimic the action of estrogen in the brain. Flavanols and anthocyanins interact with signal transmission pathways of neuron such as mitogen-activated protein kinase (MAPK) pathway and phosphoinositide 3-kinase (PI3 kinase)/Akt signal transmission stage reaction. MAPK and PI3 kinase pathways were known that memory is stored in the hippocampus and the cortex of the brain, i.e., memory and learning ability are very likely to promote by activating kinases of these pathways.

In addition, there was known that they stimulate activation of proteins such as cAMP response element binding (CREB) which is related to the expression of gene associated with memory. According to researchers at Kings College London in the UK, it was reported that ingredients contained in broccoli, potato, orange, apple and radish act in the same manner of Alzheimer's disease therapeutics and inhibit acetylcholinesterase which destroy acetylcholine (neurotransmitter).

Therefore, there is a need for natural substances derived from medicinal herbs and plants capable of treating or preventing memory and cognitive disorders by screening acetylcholinesterase inhibitors, receptor agonists and antagonists, and analyzing physiological functions to activate proteins involved in enhancement of memory and cognitive functions.

Throughout this application, several patents and publications are referenced and citations are provided in parentheses. The disclosure of these patents and publications is incorporated into this application in order to more fully describe this invention and the state of the art to which this invention pertains.

### DETAILED DESCRIPTION OF THIS INVENTION

### Technical Purposes of this Invention

The present inventors have made intensive studies to develop a safe substance to human body, especially, a plant-derived substance for enhancing memory and learning ability, effectively. As a result, the present inventors have found out that Artemisia apiaceae, Illicium verum and Lepidium apefalum used as conventional medicinal herbs are significantly effective for enhancing memory and learning ability.

Accordingly, it is an object of the present invention to provide a food composition for enhancing memory and learning ability, comprising a plant extract as an active ingredient; wherein the plant extract is one or more selected from the group consisting of an extract of Artemisia apiaceae, an extract of Illicium verum and an extract of Lepidium apefalum.

It is another object of the present invention to provide a pharmaceutical composition for preventing or treating cognitive disorders, comprising a plant extract as an active ingredient; wherein the plant extract is one or more selected from the group consisting of an extract of Artemisia apiaceae, an extract of Illicium verum and an extract of Lepidium apefalum.

It is still another object of the present invention to provide a food composition for improving cognitive disorders, comprising a plant extract as an active ingredient; wherein the plant extract is one or more selected from the group consisting of an extract of Artemisia apiaceae, an extract of Illicium verum and an extract of Lepidium apefalum.

Other objects and advantages of the present invention will become apparent from the detailed description to follow taken in conjugation with the appended claims and drawings.

### Technical Solutions of this Invention

In one aspect of the present invention, there is provided a food composition for enhancing memory and learning ability, comprising a plant extract as an active ingredient; wherein the plant extract is one or more selected from the group consisting of an extract of Artemisia apiaceae, an extract of Illicium verum and an extract of Lepidium apefalum.

In another aspect of the present invention, there is provided a pharmaceutical composition for preventing or treating cognitive disorders, comprising a plant extract as an active ingredient; wherein the plant extract is one or more selected from the group consisting of an extract of Artemisia apiaceae, an extract of Illicium verum and an extract of Lepidium apefalum.

In still another aspect of the present invention, there is provided a food composition for improving cognitive disorders, comprising a plant extract as an active ingredient; wherein the plant extract is one or more selected from the group consisting of an extract of Artemisia apiaceae, an extract of Illicium verum and an extract of Lepidium apefalum.

The present inventors have made intensive studies to develop a safe substance to human body, especially, a plant-derived substance for enhancing memory and learning ability, effectively. As a result, the present inventors have found out that Artemisia apiaceae, Illicium verum and Lepidium apefalum used as conventional medicinal herbs are significantly effective for enhancing memory and learning ability.

When the plant extract used in the present composition is obtained by treating an extraction solvent to the plant, the extract may be prepared using various extraction solvents. Preferably, the extraction solvent includes polar and non-polar solvents. The suitable polar solvent includes (i) water, (ii) alcohols (preferably, methanol, ethanol, propanol, butanol, n-propanol, iso-propanol, n-butanol, 1-pentanol, 2-butoxyethanol or ethylene glycol), (iii) acetic acid, (iv) DMFO (dimethyl formamide) and (v) DMSO (dimethyl sulfoxide). The suitable non-polar solvent includes acetone, acetonitrile, ethyl acetate, methyl acetate, fluoroalkane, pentane, hexane, 2,2,4-trimethylpentane, decane, cyclohexane, cyclopentane, diisobutylene, 1-pentene, 1-chlorobutane, 1-chloropentane, o-xylene, diisopropyl ether, 2-chloropropane, toluene, 1-chloropropane, chlorobenzene, benzene, diethyl ether, diethyl sulfide, chloroform, dichloromethane, 1,2-dichloroethane, aniline, diethylamine, ether, carbon tetrachloride and THF.

More preferably, the extraction solvent used in this invention includes (a) water, (b) absolute or hydrous lower alcohol containing 1-4 carbon atoms (methanol, ethanol, propanol, butanol, etc.), (c) mixture of lower alcohol and water, (d) acetone, (e) ethyl acetate, (f) chloroform, (g) butyl acetate, (h) 1,3-butyleneglycol, (i) hexane and (j) diethylether. Most preferably, the extraction solvent used in this invention includes water, methanol, ethanol and their combination.

The term used herein “extract” encompasses not only a crude extract but also a fraction obtained by fractionation of the crude extract. In other words, the extract from the plant includes not only the resultant of extraction using the extraction solvent described above but also the resultant of additional purifications. For instance, it could be appreciated that active fractions obtained using a variety of additional purification methods such as an ultrafiltration with defined molecular weight cut-off value and various chromatography (designed for purification dependent upon size, charge, hydrophobicity and affinity) are included in the present extracts.

The plant extract used in present invention may be powdered through additional processes such as lyophilization and spray drying.

The present composition is very effective in preventing or treating cognitive disorders as well as enhancing memory and learning ability.

The effects of the present invention are exhibited by protecting neurons and inhibiting neuronal damage, preferably acethylcholinergic neurons in cerebral base.

Acetylcholine is neurotransmitter which is secreted from nerve terminal to transmit nerve stimulus to muscle. After transmission of stimulus, it is degraded to choline and acetate by acetylcholinesterase (AChE).

According to an embodiment, the composition inhibits degradation of acethylcholine by inhibiting an activity of acetylcholinesterase to protect neurons and to prevent neuronal damage.

In addition, the present composition has an antioxidant activity. Through the antioxidant activity, the present invention exhibits effects that failures in memory and cognitive function by neuronal damages may be inhibited, and memory and learning ability may be enhanced by preventing neuronal damages.

The term used herein “reactive oxygen species (ROS)” refer to oxygen derivatives from oxygen metabolism or the transfer of free electrons, resulting in the formation of free radicals. i.e., reactive oxygen species (ROS) means to unstable state by having free radicals, whereby they have a strong activity.

When reactive oxygen species (ROS) are overproduced, they lead to oxidative stress. For that reason, they are called free radicals. Free radicals destroy the homeostasis in cells by being oxidized intracellular macromolecule (proteins, lipids, etc.) and kill cells such that fatal damages are induced in intracellular organization. In addition, they are related to aging and various causes of degenerative diseases such as cancer, muscular dysplasia, Alzheimer's disease, Parkinson's disease, ischemic disease and arteriosclerosis.

For instance, reactive oxygen species (ROS) include superoxide anion radical (O²⁻), hydrogen peroxide (H₂O₂), hydroxyl radical (OH), lipid peroxide, nitric oxide (NO), peroxinitrite (NO₃²⁻) and thiol peroxy radical (R—SO²⁻). Among them, H₂O₂ is generated by stimulus such as TGF, −1, PDGF, and EGF, and it is drawing attentions as a secondary signaling substances. Because H₂O₂ has suitable properties as signaling substances which is generated and perished in short time; H₂O₂ is relatively suitable unless it react with transition metals and less toxic. In addition, H₂O₂ is easily diffused to pass cell membranes and react to external stimulus.

According to an embodiment, the present invention has an antioxidant activity, more preferably the present invention inhibits or removes generation of reactive oxygen species and ultimately prevents neuronal damages, whereby the present composition may not only enhance memory or learning ability, but also prevent or treat cognitive disorders.

The present invention binds to NMDA (N-methyl-Daspartate) receptor to inhibit neuronal cell death, whereby neurons are protected and neuronal damages are prevented. Therefore, the present invention may not only prevent failures in memory or learning ability caused by neuronal damages, but also prevent or treat cognitive disorders by binding to NMDA (N-methyl-Daspartate) receptor.

According to an embodiment, the present invention acts as an antagonist to NMDA (N-methyl-Daspartate) receptor to prevent neuronal damages, whereby the present invention may not only enhance memory or learning ability, but also prevent or treat cognitive disorders.

The term used herein “cognitive disorders” refer to diseases that cognitive functions such as memory, perception, and problem solving are not accomplished.

The composition in the present invention may prevent and/or treat cognitive disorders. More detailed, the present invention may prevent and/or treat cognitive disorders through inhibition of the acetylcholinesterase activity, the antioxidant activity (e.g., reactive oxygen species) and the binding to NMDA (N-methyl-Daspartate) receptor.

According to an embodiment, cognitive disorders in the present invention include dementia, learning disorder, agnosia, amnesia, aphasia, apraxia or delirium, more preferably AIDS dementia complex, Binswanger's disease, dementia with Lewy Bodies, frontotemporal dementia, mild cognitive impairment, multi-infarct dementia, Pick's disease, semantic dementia, Alzheimer's dementia or vascular dementia.

The present composition may be prepared to a pharmaceutical composition.

According to an embodiment, the present composition is a pharmaceutical composition comprising (a) a pharmaceutically effective amount of the plant extract; and (b) a pharmaceutically acceptable carrier. The term used herein “pharmaceutically effective amount” refers to amount sufficient to accomplish efficacies or activities of the plant extract described above.

When the composition of the present disclosure is prepared as a pharmaceutical composition, the pharmaceutical composition of the present disclosure may comprise a pharmaceutically acceptable carrier.

The pharmaceutical composition may contain a pharmaceutically acceptable carrier. In the pharmaceutical compositions of this invention, the pharmaceutically acceptable carrier may be conventional one for formulation, including lactose, dextrose, sucrose, sorbitol, mannitol, starch, rubber arable, potassium phosphate, arginate, gelatin, potassium silicate, microcrystalline cellulose, polyvinylpyrrolidone, cellulose, water, syrups, methyl cellulose, methylhydroxy benzoate, propylhydroxy benzoate, talc, magnesium stearate, and mineral oils, but not limited to. The pharmaceutical composition according to the present invention may further include a lubricant, a humectant, a sweetener, a flavoring agent, an emulsifier, a suspending agent, and a preservative. Details of suitable pharmaceutically acceptable carriers and formulations can be found in Remington's Pharmaceutical Sciences (19th ed., 1995), which is incorporated herein by reference.

The pharmaceutical composition of this invention may be administered orally or parenterally, preferably orally.

A suitable dose of the pharmaceutical composition of the present invention may vary depending on pharmaceutical formulation methods, administration methods, the patient's age, body weight, sex, severity of diseases, diet, administration time, administration route, an excretion rate and sensitivity for a used pharmaceutical composition. Physicians of ordinary skill in the art can determine an effective amount of the pharmaceutical composition for desired treatment. Generally, the pharmaceutical composition of the present invention may be administered with a daily dose of 0.001-100 mg/kg (body weight).

According to the conventional techniques known to those skilled in the art, the pharmaceutical composition may be formulated with pharmaceutically acceptable carrier and/or vehicle as described above, finally providing several forms including a unit dose form and a multi-dose form. The formulation may be in the form of a solution in oily or aqueous medium, a suspension, a syrup, a emulsion, an extract, an elixir, a powder, a granule, a tablet or a capsule, and may further include a dispersant or stabilizer.

The present composition may be provided in a food composition.

When the composition of the present disclosure is prepared as a food composition, the food composition of the present disclosure may comprise, in addition to an extract of Artemisia apiaceae, an extract of Illicium verum or an extract of Lepidium apefalum of the present disclosure as the active ingredient, ingredients commonly added for preparation of food. For example, proteins, carbohydrates, fats, nutrients, seasoning or flavors may be added. The carbohydrate may be, for example, a sugar such as a monosaccharide, e.g., glucose, fructose, etc.; a disaccharide, e.g., maltose, sucrose, oligosaccharide, etc.; and a polysaccharide, e.g., dextrin, cyclodextrin, etc. and a sugar alcohol such as xylitol, sorbitol, erythritol, etc. The flavor may be a natural flavor [thaumatin, stevia extract (e.g., rebaudioside A, glycyrrhizin, etc.]) or a synthetic flavor (saccharin, aspartame, etc.). For example, when the food composition of the present disclosure is prepared as a drink, it may further comprise, in addition to the plant extract of the present disclosure, citric acid, liquefied fructose, sucrose, glucose, acetic acid, malic acid, fruit juices, Eucommia ulmoides extracts, jujube extracts, licorice extracts or the like.

In still another aspect of the present invention, there is provided a method for enhancing memory and learning ability, comprising administering to a subject in need thereof a composition comprising a plant extract as an active ingredient; wherein the plant extract is one or more selected from the group consisting of an extract of Artemisia apiaceae, an extract of Illicium verum and an extract of Lepidium apefalum.

In further aspect of the present invention, there is provided a method for preventing or treating cognitive disorders, comprising administering to a subject in need thereof a composition comprising a plant extract as an active ingredient; wherein the plant extract is one or more selected from the group consisting of an extract of Artemisia apiaceae, an extract of Illicium verum and an extract of Lepidium apefalum.

Since the plant extract of the present invention is described in the present composition, the common descriptions between them are omitted in order to avoid undue redundancy leading to the complexity of this specification.

### Effects of This Invention

The features and advantages of the present invention will be summarized as follows:

(i) The present invention relates to a composition for enhancing memory and learning ability and for preventing and treating cognitive disorders, comprising a plant extract as an active ingredient; wherein the plant extract is one or more selected from the group consisting of an extract of Artemisia apiaceae, an extract of Illicium verum and an extract of Lepidium apefalum.

(ii) The present invention has the effect of inhibiting neuronal damages, especially, neurons in the basal part of the cerebrum, through inhibition of acetylcholinesterase activity, the antioxidative activity (e.g., reactive oxygen species) and affinity for NMDA receptor.

(iii) The compositions of the present invention may have the effect of not only enhancing memory or learning ability by protecting neurons and preventing neuronal damages but also preventing and treating diseases caused by cognitive impairment.

(iv) In addition, the present invention is provided basic data as drug and food of the plant extract which has efficacy in enhancing memory or learning ability or efficacy for preventing and treating cognitive impairment.

### BRIEF DESCRIPTION OF THE DRAWINGS

FIG. 1 represents images of AChE inhibitory effect of Artemisia apiaceae ethanol extract (FIG. 1a), Illicium verum ethanol extract (FIG. 1b), Lepidium apefalum ethanol extract (FIG. 1c).

FIG. 2 represents images of antioxidative activity effect of Artemisia apiaceae ethanol extract (FIG. 2a), Illicium verum ethanol extract (FIG. 2b), Lepidium apefalum ethanol extract (FIG. 2c).

FIG. 3 represents images of NMDA receptor affinity effect of Artemisia apiaceae ethanol extract (FIG. 3a), Illicium verum ethanol extract (FIG. 3b), Lepidium apefalum ethanol extract (FIG. 3c).

FIG. 4 shows enhancing effect of Artemisia apiaceae ethanol extract to entering time into the dark chamber (step-through latency, STL) in scopolamine-induced memory deficit rat model.

FIG. 5 shows effect of Artemisia apiaceae ethanol extract on memory of scopolamine-treated rat.

### BEST MODE FOR CARRYING OUT THE INVENTION

The present invention will now be described in further detail by examples. It would be obvious to those skilled in the art that these examples are intended to be more concretely illustrative and the scope of the present invention as set forth in the appended claims is not limited to or by the examples.

### Example

Throughout the present specification, unless otherwise stated, “%” is used to indicate the concentration of a particular substance, solid/solid is (weight/weight) %, solid/liquid is (weight/volume) % and liquid/liquid is (volume/volume) %.

### Materials and Methods

Dried Artemisia apiaceae was purchased from a internet site (http://www.jchanbang.com) and ground by KA2610 (Jworldtech, Korea). 2 kg of Artemisia apiaceae powder was extracted with 80% ethanol (Artemisia apiaceae powder:solvent=1:10) at 95° C. for 6 hrs using COSMOS-660 (kyungseo, Korea). The ethanol extract was concentrated under reduced pressure and used as test samples.

Illicium verum and Lepidium apefalum were purchased from Dongin (China). Dried Illicium verum and Lepidium apefalum were extracted with 80% ethanol at 95° C. for 6 hrs using COSMOS-660 (kyungseo, Korea). The ethanol extracts were stored at −80° C., lyophilized to obtain powder and then used as test samples. The samples were dissolved in third-distilled water or DMSO (dimethyl sulfoxide) shortly before use and used as test liquids.

### Measurement of Inhibitory Activity to Ache (Acetylcholinesterase)

AChE (Acetylcholinesterase) inhibitory activities of the extract of Artemisia apiaceae, the extract of Illicium verum and the extract of Lepidium apefalum were measured by the method which is modified Ellman method (Ellman, G L, et al, Biochem. Pharmacol., 7:88-95, (1961)). AChE hydrolyses the acetylthiocholine to produce thiocholine. Thiocholine reacts with DTNB (5,5′-dithiobis-2-nitrobenzoic acid) to give NTB (2-nitrobenzoic acid), a yellow-colored product which absorbs at 412 nm. Therefore, AChE (Acetylcholinesterase) inhibitory activities were observed by measuring absorbance of NTB at 412 nm.

i.e., 445 μl of 0.1 M SPB (sodium phosphate buffer; pH 8.0) and 25 μl of test liquid (100 μg/ml of final concentration) were added in 500 μl cuvette. 18 μl of 0.001 M DTNB (Sigma Aldrich, St. Louis, Mo., USA) and 6 μl of 0.0075 M acetylthiocholine iodide (Sigma Aldrich, St. Louis, Mo., USA) were added and reacted at 25° C. for 4 min.

Immediately reaction, the resultant was added 6 μl of acetylcholinesterase (0.072 unit, Sigma Aldrich, St. Louis, Mo., USA), reacted in constant-temperature bath at 25° C. for 12 min and measured absorbance at 412 nm.

All experimental groups were conducted three times to obtain the average absorbance values. Effect on AChE inhibitory activity of the extract in the present invention was calculated using absorbance value of the experimental group which is added with the extract of the present invention and absorbance value of the control group which is added with 0.1 M SPB (pH 8.0) in place of the extract of the present invention, as follows:

Inhibition (%)=100−(absorbance of the control group/absorbance of the experimental group×100)

Meanwhile, the positive control group used 100 μl/ml of tacrine (Sigma Aldrich, St. Louis, Mo., USA) and eserine (Sigma Aldrich, St. Louis, Mo., USA).

### Measurement of Antioxidant Activity

Antioxidant activities of the extract of Artemisia apiaceae, the extract of Illicium verum and the extract of Lepidium apefalum were observed using antioxidant assay kit (Cayman) by measuring inhibiting level to oxidation that ABTS (2,2′-azino-di-[3-ethylbenzthiazoline silphonate]) is oxidized to ABTS⁺ by metmyoglobin. i.e., metmyoglobin reacts with hydrogen peroxide to produce ferryl myoglobin radical. Ferryl myoglobin radical oxidizes ABTS to ABTS⁺ having radical cation, a green-colored product which absorbs at 405 nm. The antioxidant existent in the extract of the present invention inhibits production of green-colored ABTS radical by suppressing the reaction by elimination of electron-donating radical, whereby it may be represented by measuring absorbance. At this time, an amount of the antioxidant in the extract of the present invention which inhibits oxidation of ABTS was represented as compared with Trolox which is tocopherol analogue.

10 μl of each extracts of Artemisia apiaceae, Illicium verum and Lepidium apefalum, 10 μl of metmyoglobin (Cayman Chemical Company, MI, USA) and 150 μl of chromogen (Cayman Chemical Company, MI, USA) were added in 96 wells plate. Then, 40 μl of hydrogen peroxide (Cayman Chemical Company, MI, USA) was added to react. After incubation for 5 min, its absorbance was measured at 405.

All experimental groups were conducted three times to obtain the average absorbance values. The antioxidant activity of the extract in the present invention was observed by measuring absorbance value of Trolox standard solution (Cayman Chemical Company, MI, USA) which is added with serial diluted Trolox in place of test sample. Then, it was represented as compared with absorbance value of the experimental group which is added with test sample.

### NMDA Receptor Affinity Test

NMDA receptor affinity was investigated by measuring the degree of inhibitory that plant extracts inhibit affinity of NMDA receptor isolated from mouse cerebrum and [³H]-glycine.

10 μl of each serial diluted extracts of Artemisia apiaceae, Illicium verum and Lepidium apefalum, 10 μl of metmyoglobin (Cayman Chemical Company, MI, USA), 180 μl of membrane isolated from mouse cerebrum and 10 μl of 300 nM [³H]-glycine were added in 96 wells plate and reacted on the ice for 40 min. After completion of reaction, the resultant was filtered using glass fiber filter (1450-421 Filtermat A) in cell harvester (MicroBeta FilterMate-96 Harvester, USA) and completely dried in the oven. After drying, the resultant was added cocktail solution and measured radioactivity using MicroBeta counter (Wallac 1450 MicroBeta counter, Perkin-Elmer, USA) such that specific binding was calculated.

All experimental groups were conducted three times to obtain the average absorbance values. 1 mM D-serine was used for calculation of non-specific binding. The specific binding of plant extract and affinity of NMDA receptor was calculated as follows:

Specific binding=Total binding−Non-specific binding

Binding (%)=((CPM [teat sample]−CPM [Non-specific binding])/(CPM [Total binding]−CPM [Non-specific binding]))×100

(* CPM: Counts per minute)

### Passive Avoidance Test and Radial Maze Test

Ethology test such as passive avoidance test and radial maze test were performed for Artemisia apiaceae. In the case of passive avoidance test, the control group, the scopolamine-treated group (3 mg/kg, i.p.), the tacrine-treated group after scopolamine treatment (10 mg/kg p.o.) and the Artemisia apiaceae extract-treated group were shocked. After 24 hrs, entering time to dark chamber (step-through latency, STL) was measured. The rote memory-enhancing effect was verified through a passive avoidance test. Furthermore, in order to verify enhancement in more specific learning ability, acquisition test in which an error frequency is measured was performed using 8-arm radial maze.

### Statistical Analysis of Data

The experimental results of the present invention were shown as the mean and standard deviation, and significance tests were performed Student's t-test using Sigma Plot (Sigma Plot).

### Result and Discussion

### Measurement of Inhibitory Activity to AChE (Acetylcholinesterase)

Acetylcholine is neurotransmitter which is secreted from nerve terminal to transmit nerve stimulus to muscle. After transmission of stimulus, it is degraded to choline and acetate by AChE.

Studies have found that cognitive disorder such as loss of memory and learning ability caused by senile dementia result from damage of acethylcholinergic neurons in cerebral base. In the present, AChE inhibitor is the most commonly researched and developed cognitive enhancer of dementia in the world. In addition, all of cognitive enhancers in dementia which are approved by FDA are the AChE inhibitors. Therefore, in the present invention, plant resources having AChE inhibitory activity were searched by evaluating enzyme activity.

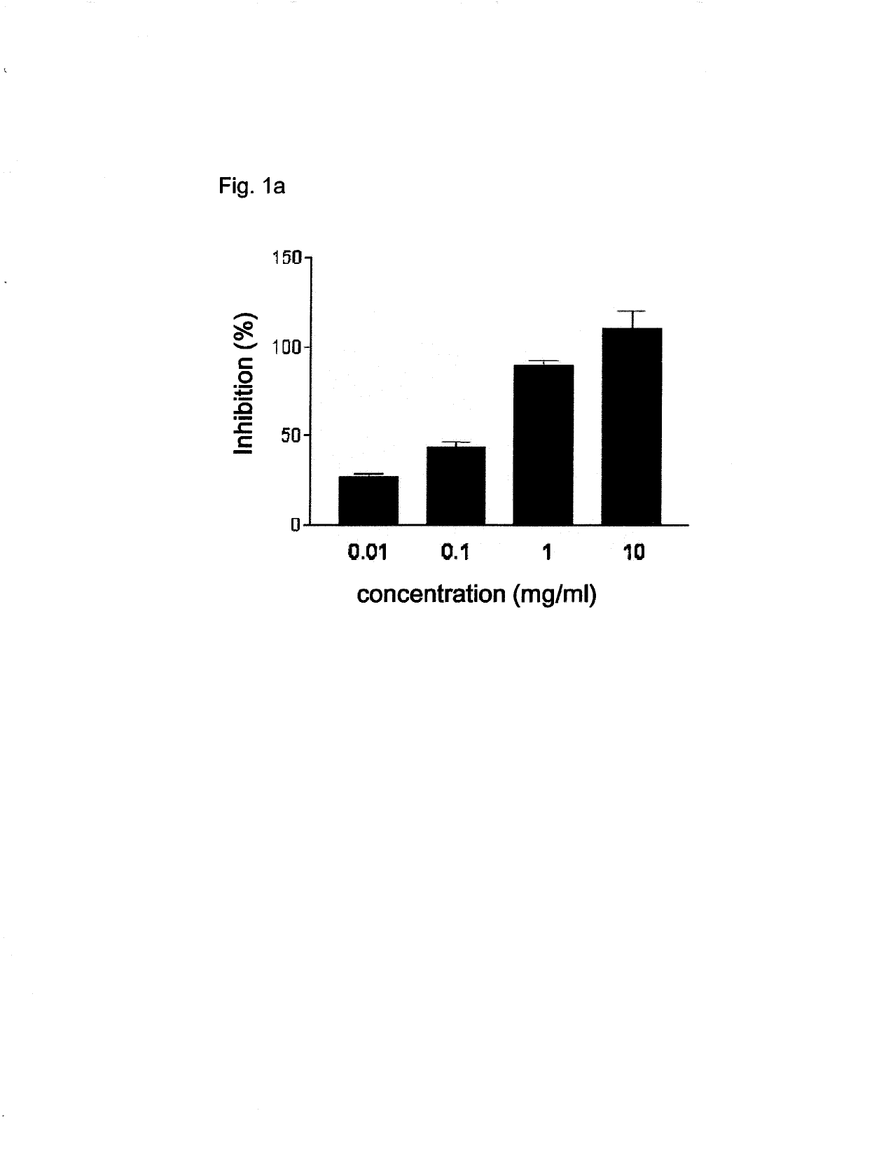

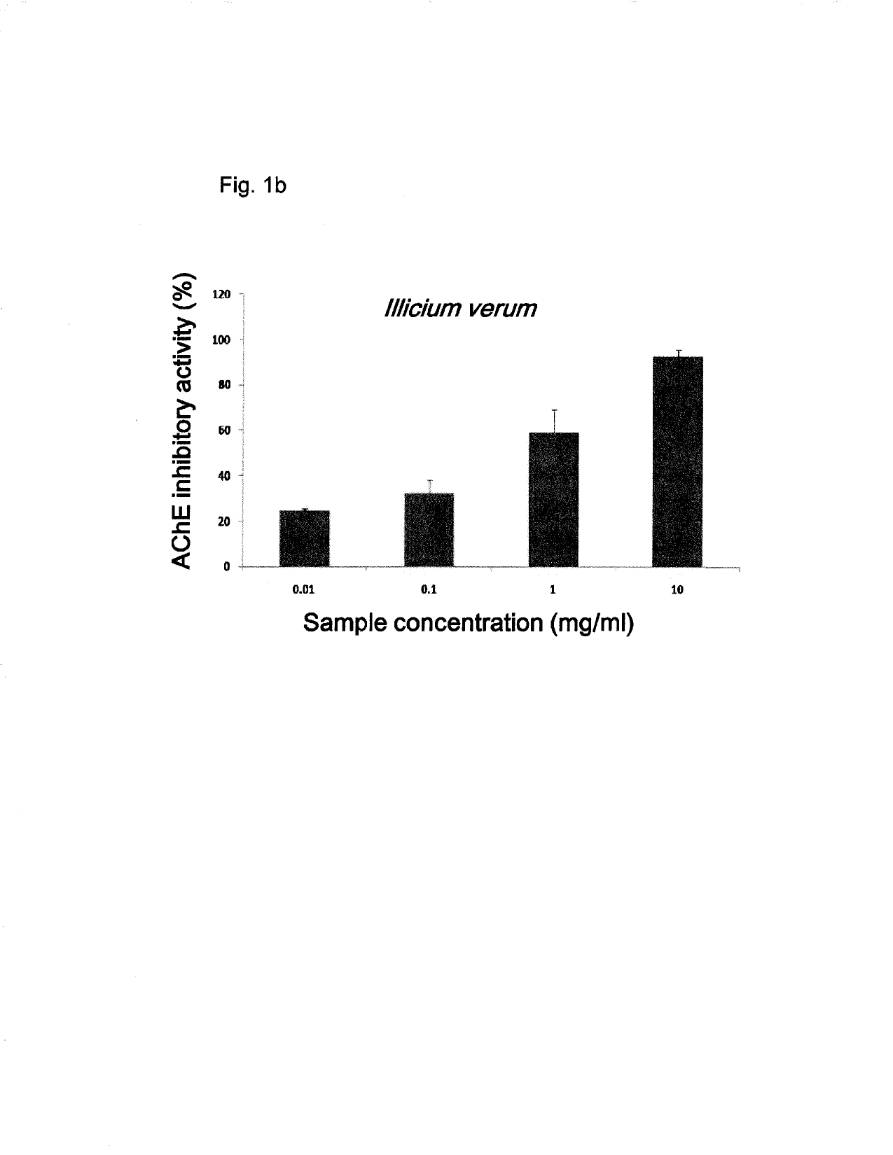

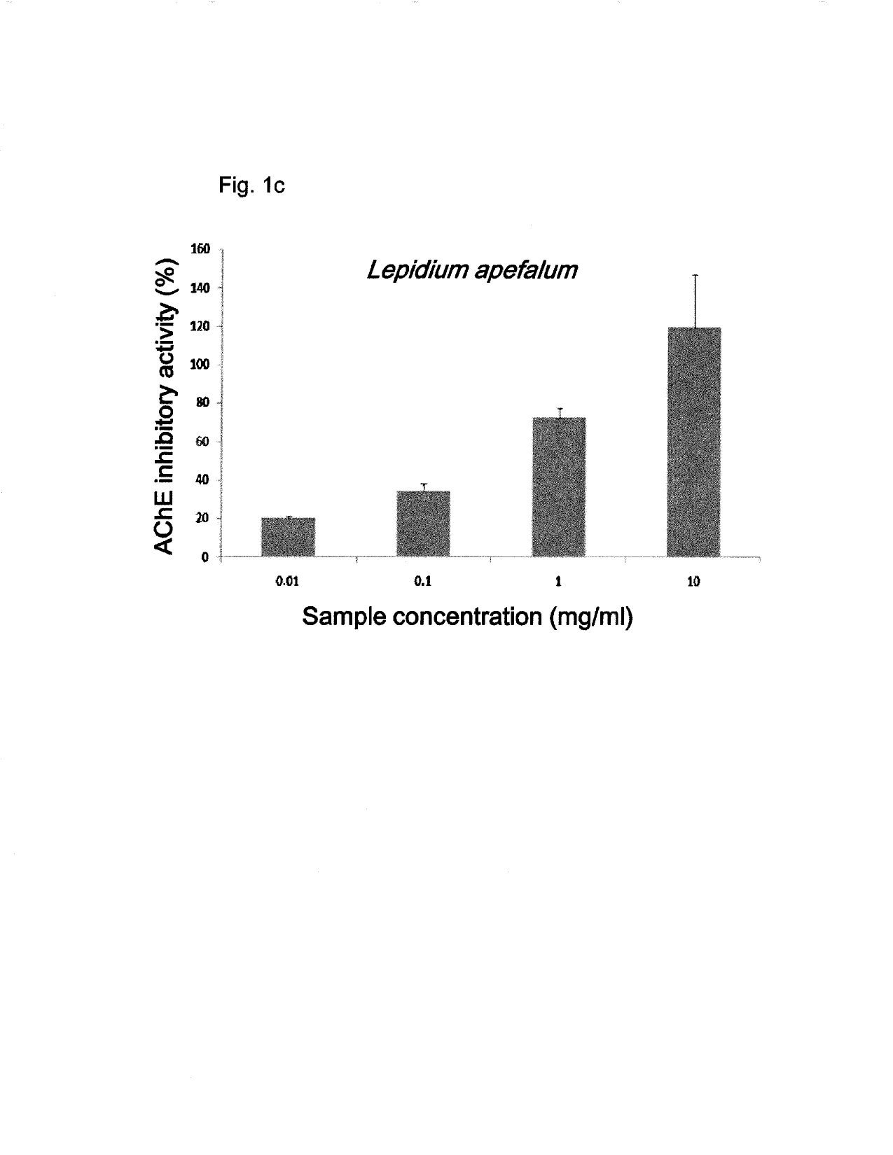

FIGS. 1a-1c show results of AChE (Acetylcholinesterase) inhibitory activities of the extract of Artemisia apiaceae, the extract of Illicium verum and the extract of Lepidium apefalum. The AChE was purified from an electric eel.

The extract of Artemisia apiaceae showed lower level of AChE inhibitory activity than 100 μg/ml of tacrine used as the positive control group. In 1 mg/ml of concentration of test sample, the extract of Artemisia apiaceae (89.44±4.05%, 80%, ethanol extract) showed higher level of AChE inhibitory activity, which is over 80%. In addition, it could be observed that all of these samples inhibited the enzyme activity depending on concentration in 0.01-10 mg/ml of concentration (FIG. 1a). Taken together, it could be determined that the extract of Artemisia apiaceae has the excellent effects of AChE inhibitory activity.

FIG. 1b shows results of AChE (Acetylcholinesterase) inhibitory activity of the extract of Illicium verum.

The extract of Illicium verum was experimented to verify AChE inhibitory activity in 0.01 mg/ml, 0.1 mg/ml, 1 mg/ml and 10 mg/ml of final concentrations, respectively. Effects of AChE (Acetylcholinesterase) inhibitory activity of the extract of Illicium verum showed 24.7%, 32.2%, 59.35% and 92.6% of inhibitory activity, respectively. The inhibitory activities were increased depending on concentrations of the extract. Especially, the extract of Illicium verum showed 59.35% of inhibitory effect in 1 mg/ml of concentration of test sample and it could be determined that the extract of Illicium verum has the excellent effects of AChE inhibitory activity.

FIG. 1c shows results of AChE (Acetylcholinesterase) inhibitory activity of the extract of Lepidium apefalum in comparison with tacrine used as the positive control group. The extract of Lepidium apefalum was experimented to verify AChE inhibitory activity in 0.01 mg/ml, 0.1 mg/ml, 1 mg/ml and 10 mg/ml of final concentrations, respectively. Effects of AChE (Acetylcholinesterase) inhibitory activity of the extract of Lepidium apefalum showed 20.2%, 34.4%, 72.4% and 120% of inhibitory activity, respectively. The inhibitory activities were increased depending on concentrations of the extract. Especially, the extract of Lepidium apefalum showed 72.4% of inhibitory effect in 1 mg/ml of concentration of test sample and it could be determined that the extract of Lepidium apefalum has the excellent effects of AChE inhibitory activity.

### Measurement of Antioxidant Activity

Reactive oxygen species have been reported that they attack cell lipids, proteins and DNA to produce lipoperoxide and oxidized protein and to accelerate aging, and they cause oxidative damage and destruction of brain cells in the brain tissue to induce dementia. Therefore, to remove reactive oxygen species is considered as important in the prevention and treatment of dementia.

Typically, since reactive oxygen species occur serially through the autooxidation reaction, antioxidants terminate the reaction by donating a hydrogen atom to radical which is generated during autooxidation reaction in the body.

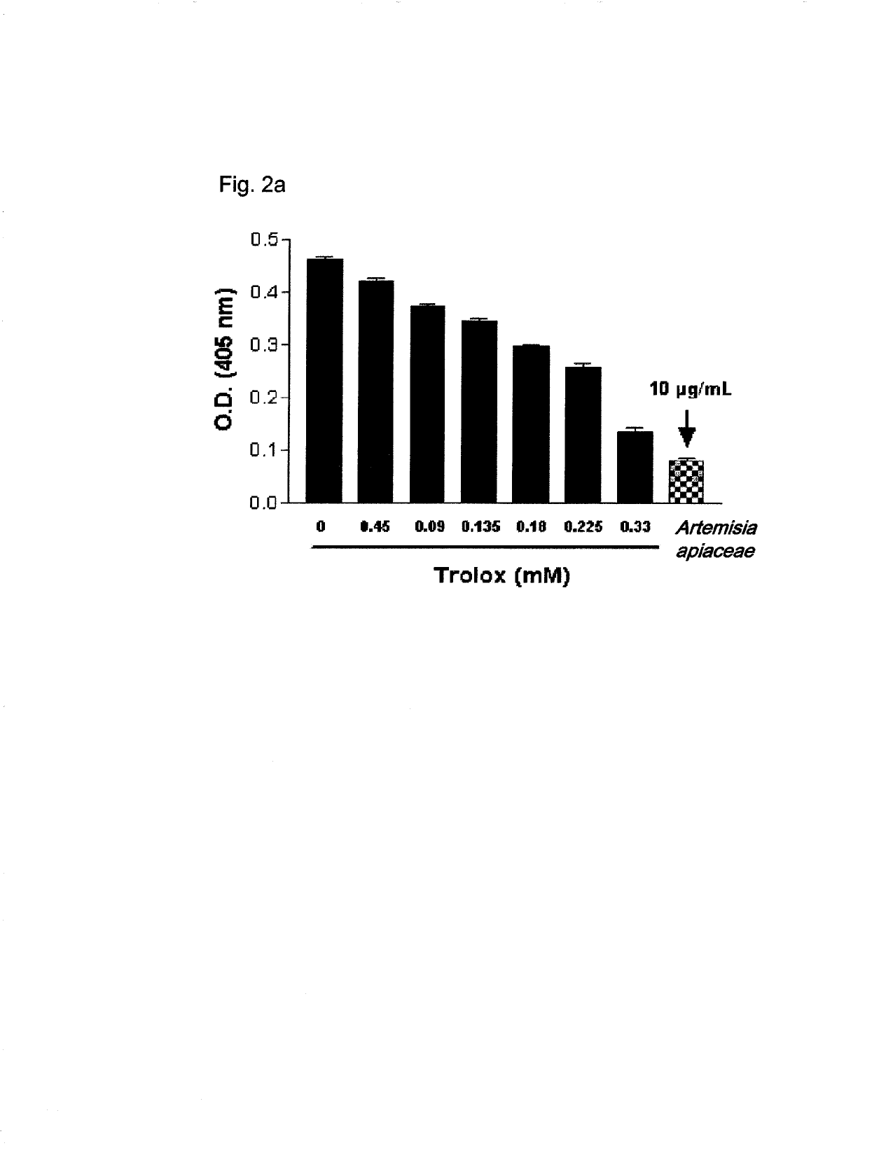

As mentioned above, antioxidants inhibit oxidation by removing radical in electron transport system. Accordingly, the present inventors have made intensive studies to search natural substances for enhancing memory by measuring the antioxidant activity of the ethanol extract. Antioxidant activities of the plant extracts were measured using antioxidant assay kit (Cayman) which measure total antioxidant activity and FIGS. 2a-2c shows the results.

As a result of antioxidant activity measurement, the extract of Artemisia apiaceae increased antioxidant activity depending on concentrations in 10-100 μg/ml of concentration. Especially, the extract of Artemisia apiaceae (effect of more than 0.33 mM Trolox) showed higher level of antioxidant activity than Trolox as the positive control group in 10 μg/ml of concentration (FIG. 2a).

The extract of Illicium verum showed lower level of O.D value than than 0.225 mM of Trolox as standard material, whereby it could be determined that the extract of Illicium verum has the excellent effect of antioxidant activity (FIG. 2b).

The extract of Lepidium apefalum showed lower level of O.D value than 0.225 mM of Trolox, whereby it could be determined that the extract of Lepidium apefalum has the excellent effect of antioxidant activity (FIG. 2c).

These results showed that the present composition may be useful as substance for enhancing memory.

### NMDA Receptor Affinity Test

Recently research to control dementia by preventing damages of brain cells by various excitatory amino acids such as glutamate and NMDA (N-methyl-D-aspartate) has been noticed.

NMDA receptor acts as the most important Ca²⁺ channel in neurons to stimulate influx of Ca²⁺ into neuron by various NMDA agonists, whereby neuronal apoptosis is induced. Accordingly, the present inventors have made intensive studies to search NMDA receptor antagonist having excellent effect from natural substances by measuring affinity between plant extract and NMDA receptor.

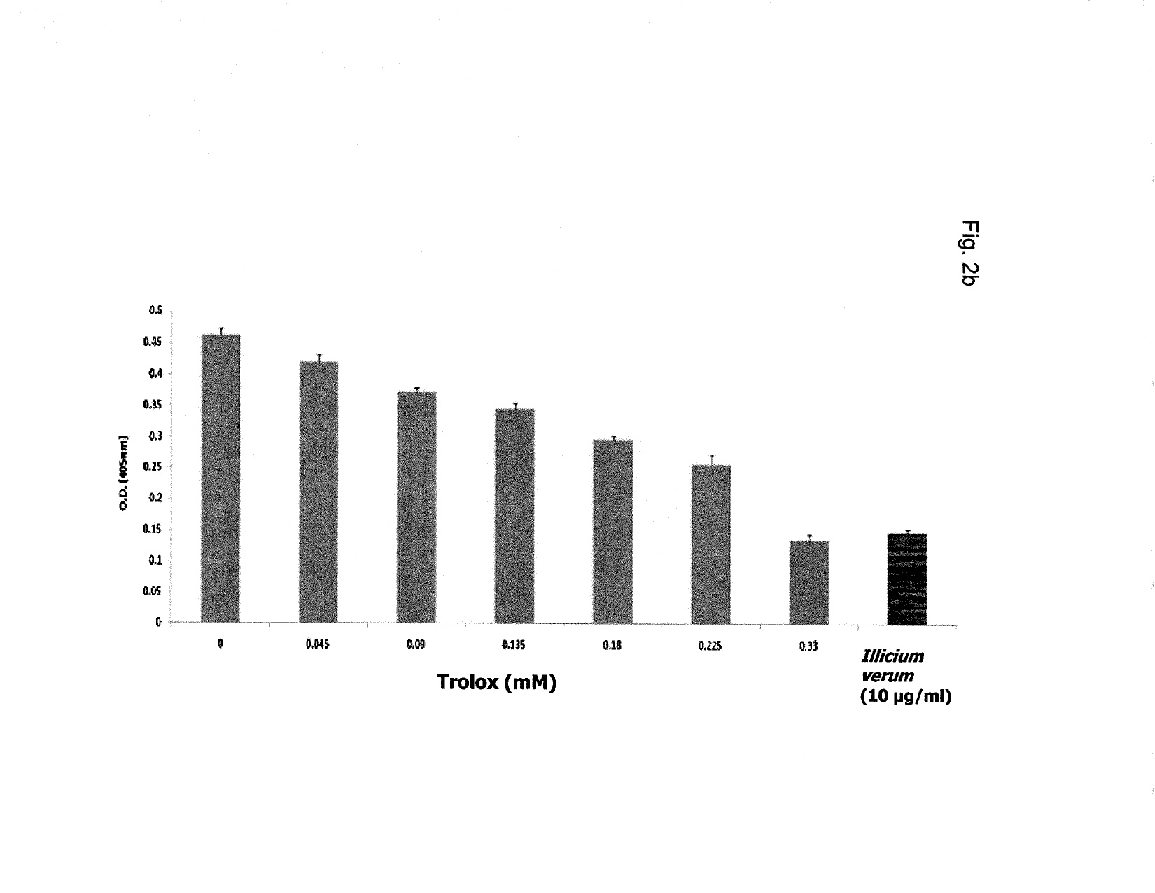

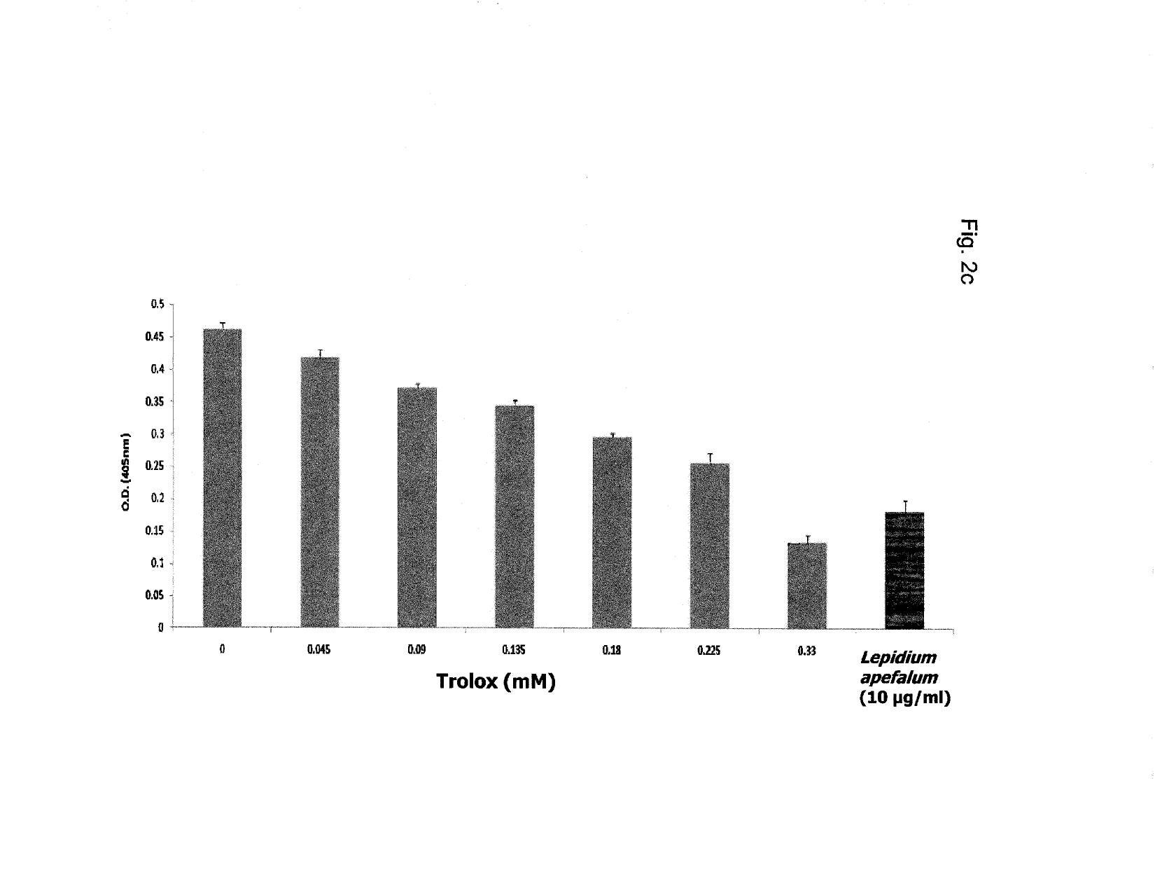

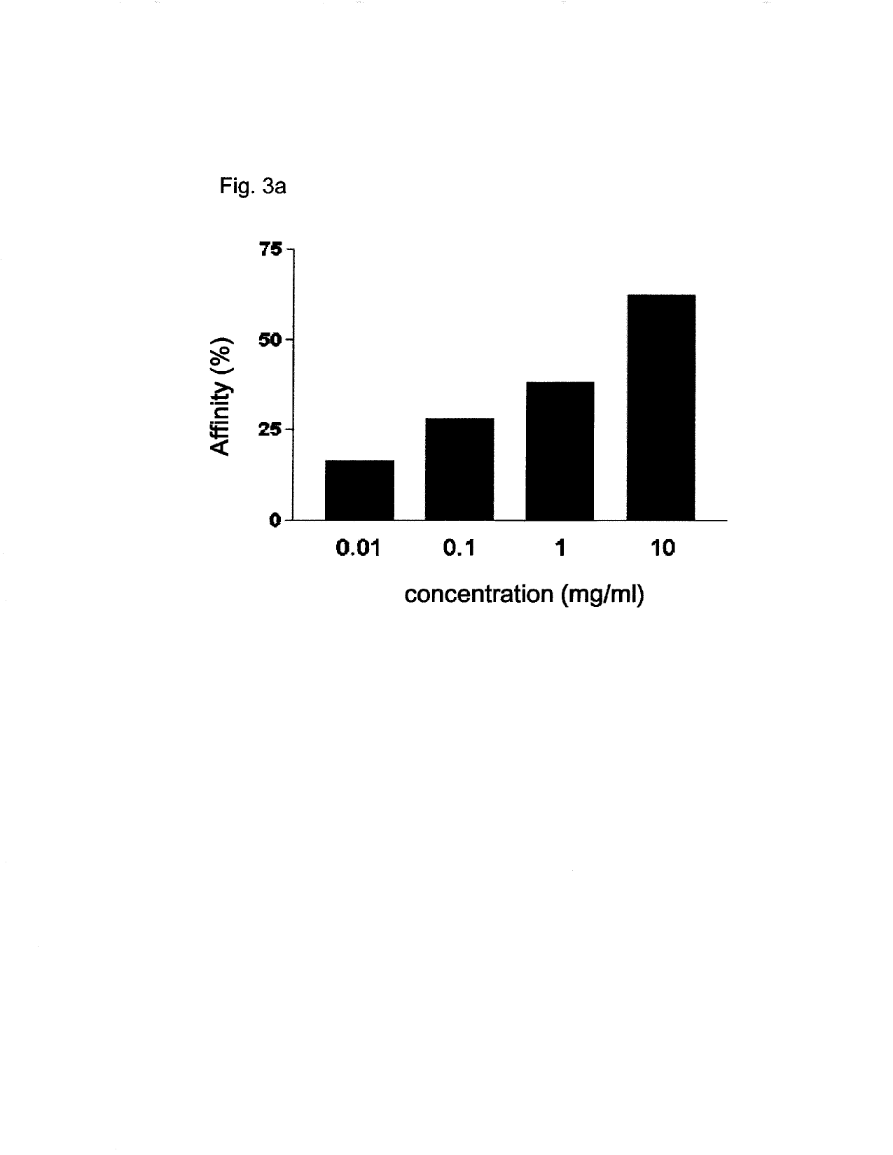

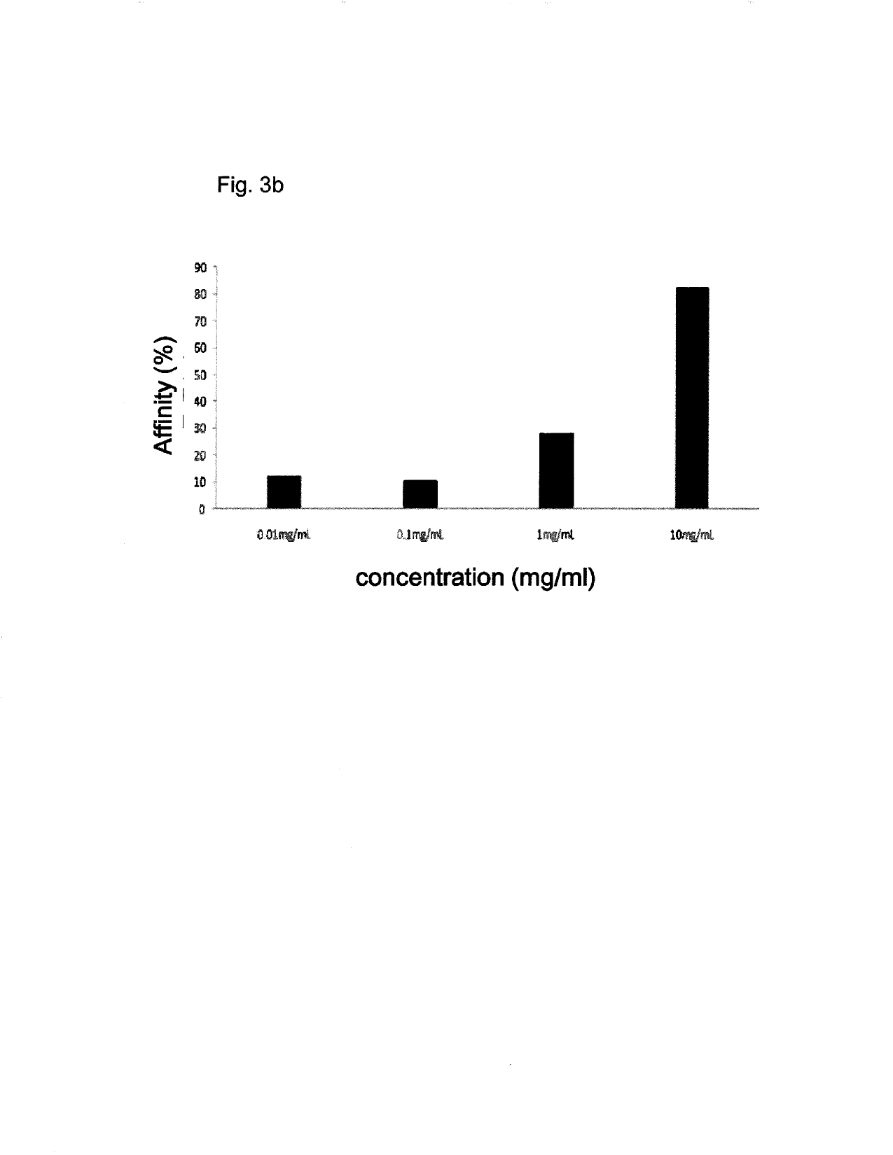

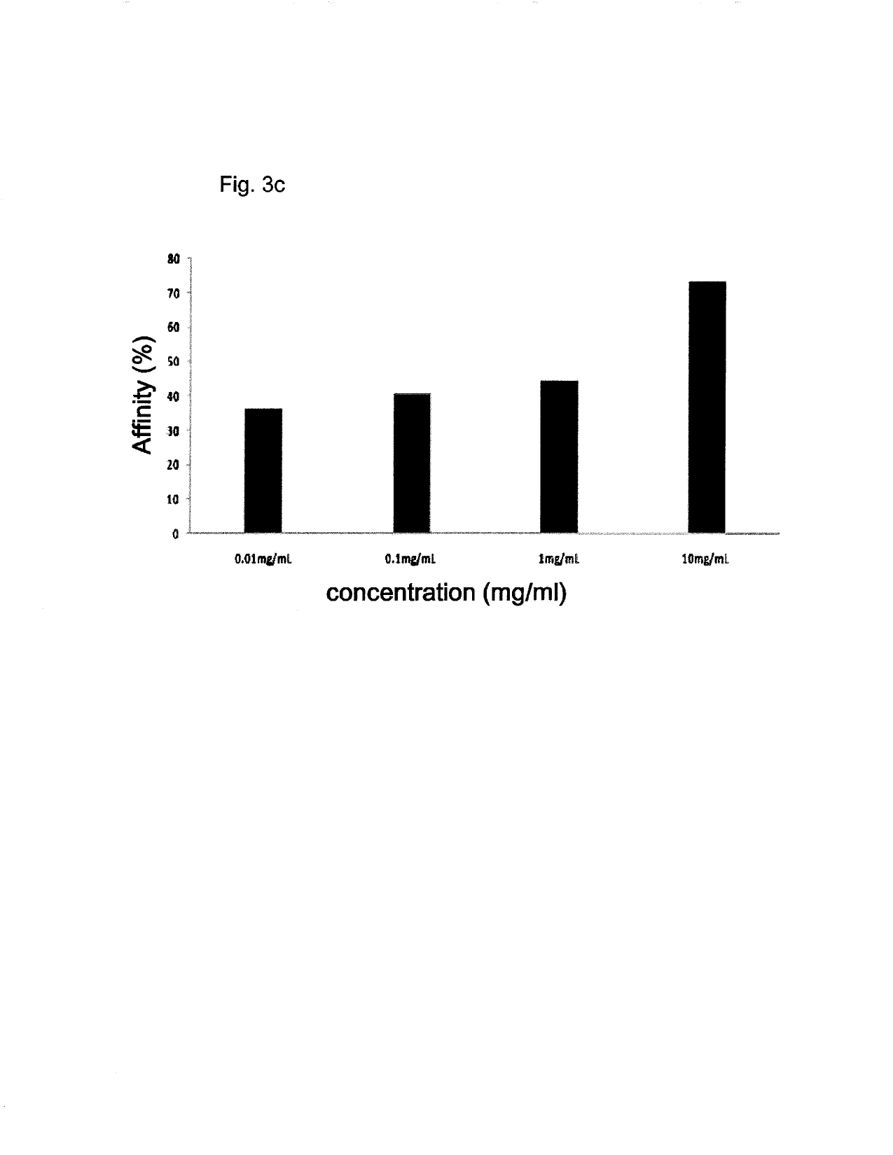

The affinity to NMDA receptor was investigated in which the extract of Artemisia apiaceae, the extract of Illicium verum or the extract of Lepidium apefalum inhibits binding interactions between NMDA receptor isolated from mouse cerebrum and [³H]-glycine known as a selective ligand to its glycine binding site. FIG. 3 showed results that each the present plant extract was diluted to 0.01, 0.1, 1 and 10 mg/ml of concentration to verify affinity to the receptor. All of the present extracts showed affinity depending on concentrations (FIGS. 3a-3c). The extract of Artemisia apiaceae (62.21% binding) showed more than 60% of the binding affinity to NMDA receptor in 10 mg/ml of concentration. The extract of Illicium verum inhibited affinity to receptor by 82.16% in 10 mg/ml of concentration. The extract of Lepidium apefalum showed 36.1, 40.5, 44.1 and 73.3% of binding affinity to NMDA receptor in 0.01, 0.1, 1 and 10 mg/ml of concentration, respectively. Especially, the extract of Lepidium apefalum inhibited affinity to receptor by 44.1% in 10 mg/ml of concentration.

It would be appreciated that researches for searching affinity to glycine binding site of NMDA receptor of natural plant based on these results may be important indicator to use in research and development for therapeutic agent of dementia in the future.

Memory Improvement Effect on Extract of Artemisia apiaceae Using Passive Avoidance Test

Ethology test such as passive avoidance test and radial maze test were performed for Artemisia apiaceae. In the passive avoidance test, the control group showed significantly increased entering time into the dark chamber as compared with the training test, by memorizing the electric shock received before 24 hrs. The scopolamine-treated group (3 mg/kg, i.p.) did not memorize the electric shock received before 24 hrs by memory loss and showed significantly decreased entering time into the dark chamber as compared with the control group. Meanwhile, the tacrine-treated group after scopolamine treatment (10 mg/kg p.o.) showed significantly increased entering time into the dark chamber. For 5 weeks the Artemisia apiaceae extract-treated group showed significantly decreased entering time into the dark chamber (step-through latency, STL) as compared with the scopolamine-treated group. Therefore, it could be determined that the ethanol extract of Artemisia apiaceae has the excellent effect for recovering memory which is damaged by scopolamine.

Memory Improvement Effect on Extract of Artemisia apiaceae Using Radial Maze Test

The enhancement effect in the rote memory verified by the passive avoidance test was further analyzed for higher-level memory (spatial memory) using a radial maze, thereby investigating the effects of the extract of Artemisia apiaceae on both learning ability and memory.

In the acquisition test by measuring the error frequency, the normal group (1.6) showed lower error frequency than the negative control group (3.25). The tacrine-treated group (1.75) was used as the positive control group. Both the tacrine-treated group and the Artemisia apiaceae extract-treated group showed significantly decreased error frequency as compared with the scopolamine-treated group.

It has been well known that the failures in memory and cognitive function are closely related with cholinergic nervous system. The previous reports described that scopolamine as an antagonist to muscarinic receptor in postsynapse inhibits binding between acetylcholine and muscarinic receptor to block information transmission temporarily, resulting in impairment in memory and learning ability. It is very likely that the extracts used in this Example inhibit the action of scopolamine to interrupt binding between acetylcholine and muscarinic receptor, thereby dramatically preventing failures in memory and cognitive function.

Having described a preferred embodiment of the present invention, it is to be understood that variants and modifications thereof falling within the spirit of the invention may become apparent to those skilled in this art, and the scope of this invention is to be determined by appended claims and their equivalents.

1-9. (canceled)

10. A method for enhancing memory and learning ability, comprising administering to a subject in need thereof a composition comprising a plant extract as an active ingredient; wherein the plant extract is one or more selected from the group consisting of an extract of Artemisia apiaceae, an extract of Illicium verum and an extract of Lepidium apefalum.

11. A method for preventing or treating cognitive disorders, comprising administering to a subject in need thereof a composition comprising a plant extract as an active ingredient; wherein the plant extract is one or more selected from the group consisting of an extract of Artemisia apiaceae, an extract of Illicium verum and an extract of Lepidium apefalum.

12. The method according to claim 10, wherein the composition inhibits an activity of acetylcholinesterase.

13. The method according to claim 10, wherein the composition has an antioxidant activity.

14. The method according to claim 13, wherein the composition inhibits or removes generation of reactive oxygen species.

15. The method according to claim 10, wherein the composition acts as an antagonist to NMDA (N-methyl-Daspartate) receptor.

16. The method according to claim 11, wherein the cognitive disorders are dementia, learning disorder, agnosia, amnesia, aphasia, apraxia or delirium.

17. The method according to claim 11, wherein the cognitive disorders are AIDS dementia complex, Binswanger's disease, dementia with Lewy Bodies, frontotemporal dementia, mild cognitive impairment, multi-infarct dementia, Pick's disease, semantic dementia, Alzheimer's dementia or vascular dementia.

18. The method according to claim 11, wherein the composition inhibits an activity of acetylcholinesterase.

19. The method according to claim 11, wherein the composition has an antioxidant activity.

20. The method according to claim 11, wherein the composition acts as an antagonist to NMDA (N-methyl-Daspartate) receptor.
